(19) United States
(12) Patent Application Publication
(10) Pub. No.: US 2023/0226540 A1
(43) Pub. Date: Jul. 20, 2023

(54) TEST CONTAINER, TEST DEVICE, AND NUCLEIC ACID TEST METHOD

(71) Applicant: FUJIFILM Corporation, Tokyo (JP)

(72) Inventors: Takeshi HAMA, Kanagawa (JP); Hiroyasu ISHII, Kanagawa (JP)

(73) Assignee: FUJIFILM Corporation, Tokyo (JP)

(21) Appl. No.: 18/190,284

(22) Filed: Mar. 27, 2023

Related U.S. Application Data
(63) Continuation of application No. PCT/JP2021/030484, filed on Aug. 20, 2021.

(30) Foreign Application Priority Data
Sep. 29, 2020 (JP) 2020-163979

(51) Int. Cl. B01L 3/00; C12Q 1/6844

(52) U.S. Cl. CPC B01L 3/502; C12Q 1/6844; B01L 2200/10; B01L 2300/0816; B01L 2200/16; B01L 2300/1805; B01L 2300/087; B01L 2300/047; B01L 2300/042; B01L 2200/0689

(57) ABSTRACT

A test container includes an inlet, a first storage portion, a second storage portion, a first flow channel that connects the first storage portion to the second storage portion, a first cylinder of which one end is connected to the first storage portion via a second flow channel and the other end is open to an outside, a second cylinder of which one end is connected to the second storage portion via a third flow channel and the other end is open to an outside, a first plug provided in the first cylinder, and a second plug provided in the second cylinder. An internal space including the first storage portion, the second storage portion, the first flow channel, the second flow channel, and the third flow channel is capable of being pressurized in a case where the first plug and the second plug are pressed and moved from the outside.

### CROSS-REFERENCE TO RELATED APPLICATIONS

This application is a continuation of International Application No. PCT/JP2021/030484, filed on Aug. 20, 2021, which claims priority from Japanese Patent Application No. 2020-163979, filed on Sep. 29, 2020. The entire disclosure of each of the above applications is incorporated herein by reference.

### BACKGROUND

### 1. Technical Field

The present invention relates to a test container, a test device, and a nucleic acid test method.

### 2. Related Art

In a technique of genetic diagnosis, a technique for amplifying a small amount of nucleic acid included in a specimen is being studied. Examples of a nucleic acid amplification method include a polymerase chain reaction (PCR) method, a loop-mediated isothermal amplification (LAMP) method, and the like.

In a nucleic acid amplification test, a nucleic acid is extracted from a specimen and a specimen solution including the nucleic acid is then mixed with an amplification reagent for amplifying a specific nucleic acid sequence (a target deoxyribonucleic acid (DNA) or a ribonucleic acid (RNA), hereinafter, collectively referred to as a target DNA) that is an object to be detected. After that, an amplification step for the target DNA is performed and whether or not the target DNA is present is then determined to perform a test for determining whether or not the target DNA as a nucleic acid to be detected is included in the specimen.

A test using a nucleic acid amplification method, such as a PCR method, is currently also used for tests for determining whether or not a person is infected with influenza, novel coronavirus, and the like. A demand for the point of care testing (POCT) is increasing for quick diagnosis, and there is a demand for a test device that can easily amplify a nucleic acid.

A micro-flow channel chip that can be used in POCT is proposed in JP2018-197694A. JP2018-197694A discloses a micro-flow channel chip. The micro-flow channel chip comprises a syringe that stores air used to push out a specimen solution into a reaction space and a syringe that stores a reagent caused to react with the specimen solution, and the specimen solution and the reagent can be mixed with each other in the micro-flow channel chip.

For example, the amplification of a target DNA using PCR is realized by the repetition of a step of dissociating a double-stranded DNA into a single-stranded DNA at a high temperature (thermal denaturation step), a step of lowering a temperature and binding a primer to the single-stranded DNA (annealing step), and a step of newly synthesizing a double-stranded DNA by polymerase using the single-stranded DNA as a template (elongation step). In a case where a target is an RNA, the target is amplified using reverse transcription (RT)-PCR. Examples of a temperature cycle of the thermal denaturation step, the annealing step, and the elongation step include 20 to 30 repetitions of one cycle of the thermal denaturation step performed for 1 minute at a temperature of 94° C., the annealing step performed for 1 minute at a temperature of 50 to 60° C., and the elongation step performed for 1 to 5 minutes at a temperature of 72° C. Further, the LAMP method is a method of causing an amplification reaction to proceed in a state where a constant temperature of about 65° C. is maintained. As described above, in general, a nucleic acid amplification reaction includes a step of heating a liquid in which a specimen solution and an amplification reagent are mixed with each other. In this case, a problem that a long time is required for the amplification step due to the inhibition of the amplification step, sufficient amplification cannot be performed, or the like is likely to occur in a case where gas contained in the specimen solution and the reagent foams due to heating treatment. In POCT application, it is required to shorten a time required for a test and to improve test accuracy.

The use of the micro-flow channel chip, which is disclosed in JP2018-197694A, for a nucleic acid amplification test is not described. For this reason, problems occurring in a reaction including a heating step and solutions to the problems are not described at all.

### SUMMARY

A technique of the present disclosure has been made in consideration of the above-mentioned circumstances, and an object of the technique of the present disclosure is to provide a test container, a test device, and a nucleic acid test method that allow a test to be performed with high test accuracy in a test including a heating step.

A test container of the present disclosure comprises: an inlet into which a specimen solution is to be put; an attachable and detachable lid part that covers the inlet; a first storage portion that is provided such that the inlet is an end surface of an opening and that stores a specimen solution added dropwise from the inlet; a second storage portion that is capable of storing liquid and causes the specimen solution and a reagent to react with each other; a first flow channel that connects the first storage portion to the second storage portion; a first cylinder of which one end is connected to the first storage portion via a second flow channel and the other end is open to an outside; a second cylinder of which one end is connected to the second storage portion via a third flow channel and the other end is open to an outside; a first plug that is provided to be movable in the first cylinder; and a second plug that is provided to be movable in the second cylinder. An internal space including the first storage portion, the second storage portion, the first flow channel, the second flow channel, and the third flow channel is capable of being pressurized in a case where the first plug and the second plug are pressed and moved from the outside.

In the test container of the present disclosure, the internal space may be hermetically sealed by the first plug and the second plug, and the second plug may be moved from the internal space to the outside in the second cylinder while being interlocked with movement of the first plug in a case where the first plug is pressed from the outside and moved toward the internal space in the first cylinder.

In the test container of the present disclosure, an air vent may be provided at a part of the second cylinder and the internal space may be capable of being pressurized in a case where the second plug is moved closer to the internal space than the air vent.

It is preferable that the test container of the present disclosure further comprises a purification chamber provided in a middle of the first flow channel and removing impurities in the specimen solution.

In the test container of the present disclosure, the reagent may be stored in the second storage portion.

The test container of the present disclosure may further comprise a third storage portion that is provided in a middle of the first flow channel and stores the reagent.

In the test container of the present disclosure, it is preferable that the third storage portion is provided between the purification chamber and the second storage portion in the middle of the first flow channel.

The test container of the present disclosure may further comprise a stirring flow channel that is provided between the third storage portion and the second storage portion and facilitates mixture of the specimen solution and the reagent.

In the test container of the present disclosure, it is preferable that a bottom surface of the second storage portion is formed of a film.

In the test container of the present disclosure, the reagent may include an amplification reagent that amplifies a specific nucleic acid sequence and a probe for determination of a nucleic acid sequence.

A test device of the present disclosure comprises: the test container of the present disclosure; and a pressing machine including a first pressing unit that presses the first plug positioned in the first cylinder of the test container from the outside and a second pressing unit that presses the second plug positioned in the second cylinder from the outside. In a case where the first plug is pressed and moved toward the internal space by the first pressing unit, the specimen solution stored in the first storage portion of the test container is fed to the second storage portion.

It is preferable that the test device of the present disclosure further comprises a first heating unit that is provided at a position allowing the first heating unit to be in contact with a bottom surface of the second storage portion of the test container and heats liquid stored in the second storage portion.

The test device of the present disclosure may further comprise a second heating unit that is provided at a position allowing the second heating unit to be in contact with a bottom surface of the first storage portion of the test container and heats liquid stored in the first storage portion.

The test device of the present disclosure may further comprise a detection unit that detects whether or not an object to be detected is included in the specimen solution in the second storage portion.

In the test device of the present disclosure, the reagent in the test container may include an amplification reagent that amplifies a specific nucleic acid sequence and a fluorescent probe for determination of a nucleic acid sequence, and the detection unit may include an excitation light source that irradiates liquid stored in the second storage portion with excitation light for exciting the fluorescent probe, and a photodetector that detects fluorescence emitted from the fluorescent probe excited by irradiation with the excitation light.

A nucleic acid test method of the present disclosure comprises: immersing a specimen, which is collected from a living body using a collection tool, in a nucleic acid extraction solution to extract a nucleic acid from the specimen; putting liquid, which includes the nucleic acid, as the specimen solution from the inlet of the test container; hermetically sealing the inlet by the lid part; feeding the specimen solution, which is stored in the first storage portion, to the second storage portion by the pressing machine; amplifying the specific nucleic acid sequence by controlling a temperature of mixed liquid of the specimen solution and the reagent in the second storage portion; irradiating the mixed liquid with the excitation light and detecting fluorescence generated from the fluorescent probe using the photodetector; and determining whether or not the specific nucleic acid sequence is present.

According to the test container, the test device, and the nucleic acid test method of the present disclosure, it is possible to perform a test with high test accuracy.

### BRIEF DESCRIPTION OF THE DRAWINGS

FIG. 1 is a plan view schematically showing a test container according to a first embodiment.

FIG. 2 is a perspective view of the test container.

FIG. 3A shows a cross section taken along line 3A-3A of FIG. 1, FIG. 3B shows a cross section taken along line 3B-3B of FIG. 1, and FIG. 3C shows a cross section taken along line 3C-3C of FIG. 1.

FIG. 4 is a diagram illustrating a liquid feeding method in the test container.

FIG. 5 is a diagram illustrating a liquid feeding method in a test container of Design Change Example 1.

FIG. 6 is a diagram illustrating a pressurization method in the test container of Design Change Example 1.

FIG. 7 is a plan view schematically showing a test container of Design Change Example 2.

FIGS. 8A and 8B are enlarged views of a second plug of the test container.

FIG. 9 is a plan view schematically showing a test container according to a second embodiment.

FIG. 10 is a plan view schematically showing a test container 5 according to a third embodiment.

FIG. 11 is a plan view schematically showing a test container 6 according to a fourth embodiment.

FIG. 12 is a partially exploded perspective view of the test container 6.

FIG. 13A shows a cross section taken along line 13A-13A of FIG. 11, FIG. 13B shows a cross section taken along line 13B-13B of FIG. 11, FIG. 13C shows a cross section taken along line 13C-13C of FIG. 11, FIG. 13D shows a cross section taken along line 13D-13D of FIG. 11, and FIG. 13E shows a cross section take along line 13E-13E of FIG. 11.

FIG. 14 is a plan view schematically showing a test container according to a fifth embodiment.

FIG. 15 is a diagram showing the schematic configuration of a test device according to an embodiment.

FIG. 16A is a plan view showing a positional relationship between the test container and a pressing machine of the test device and FIG. 16B is a cross-sectional view taken along line 16B-16B of FIG. 16A.

FIG. 17 is a diagram illustrating a test method.

### DETAILED DESCRIPTION

Embodiments of the present disclosure will be described in detail below with reference to the drawings. The scale and the like of each component shown in the drawings are appropriately changed from the actual ones.

“Test Container According to First Embodiment”

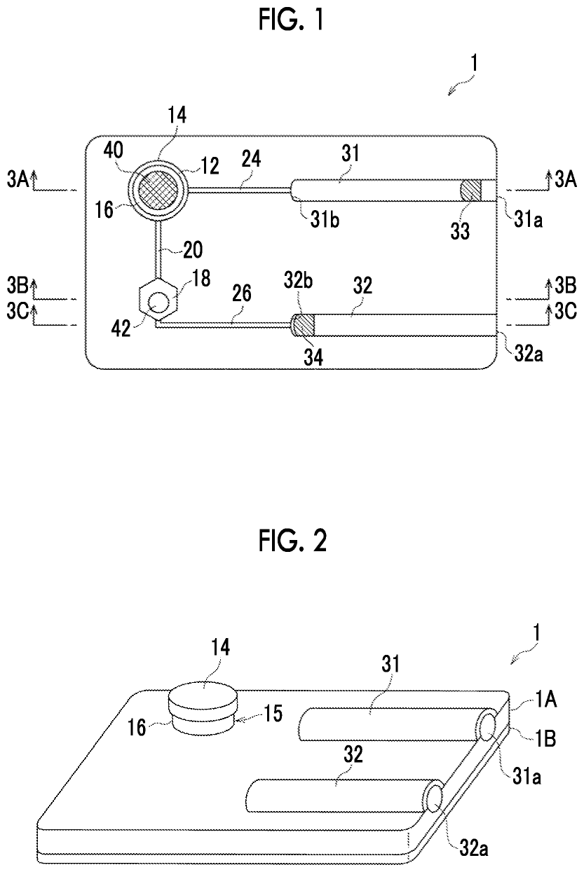

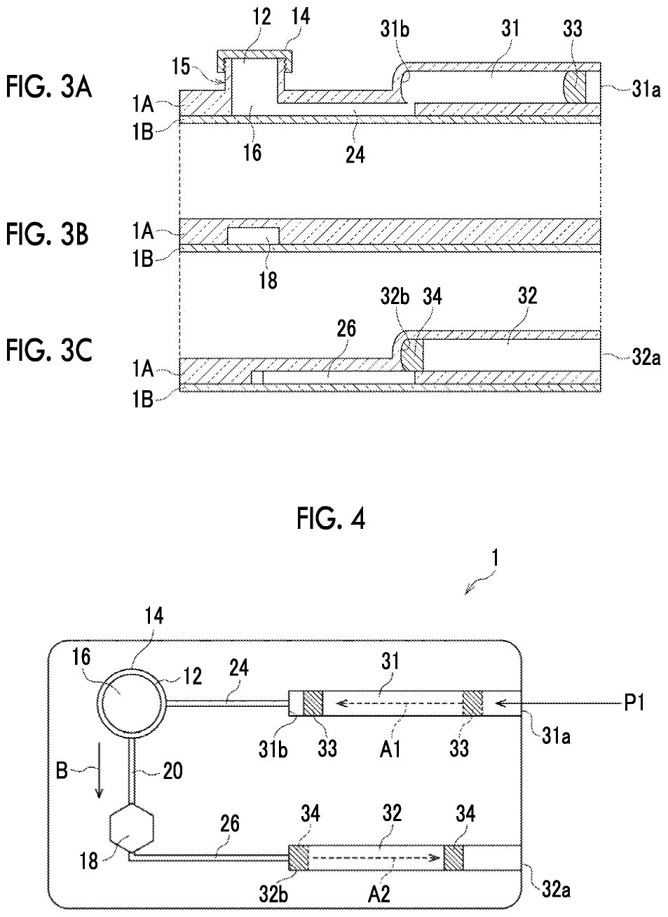

FIG. 1 is a plan view schematically showing a test container 1 according to a first embodiment, and FIG. 2 is a perspective view of the test container 1 shown in FIG. 1. Further, among FIGS. 3A, 3B, and 3C, FIG. 3A shows a cross section taken along line 3A-3A of FIG. 1, FIG. 3B shows a cross section taken along line 3B-3B of FIG. 1, and FIG. 3C shows a cross section taken along line 3C-3C of FIG. 1.

The test container 1 according to the present embodiment is a cartridge for a nucleic acid test that has a card-like appearance and includes a flow channel structure therein. The test container 1 is used to detect whether or not an object to be detected is included in a specimen by amplifying a specific nucleic acid sequence included in the specimen, which includes the specific nucleic acid sequence as the object to be detected, such that the specific nucleic acid sequence can be detected. Specifically, the test container 1 is used to test whether or not a person is infected with an infectious disease, such as influenza. The test container 1 has substantially the same plane size as a credit card and a thickness of about 1 cm.

The test container 1 includes a body member 1A in which a recessed portion and a hole portion forming a part of the flow channel structure including flow channels and storage portions are formed, and a bottom member 1B that forms a bottom surface of the flow channel.

A publicly known resin molding plastic material can be used without particular limitation as long as being used as a material of the body member 1A. However, polycarbonate, polypropylene, cycloolefin, or a silicone resin is preferable from the viewpoint of heat resistance and transparency.

The bottom member 1B is formed of, for example, a thin plate or a film. A publicly known resin molding plastic material can be used without particular limitation as long as being used as a material of the bottom member 1B. However, the same material as the body member 1A is preferable from the viewpoint of adhesion to the body member 1A.

This test container 1 comprises an inlet 12, a lid part 14, a first storage portion 16, a second storage portion 18, a first flow channel 20, a first cylinder 31, a second cylinder 32, a first plug 33, and a second plug 34. The test container 1 further comprises a second flow channel 24 and a third flow channel 26.

The inlet 12 is an opening into which a specimen solution 40 is to be put. The lid part 14 is a lid part that covers the inlet 12 and that can be attached to and detached from the opening of the inlet 12. In the present embodiment, the lid part 14 is formed to be screwable with a tubular portion 15 forming the inlet 12. A method of attaching and detaching the lid part 14 is not particularly limited, and the lid part 14 and the tubular portion 15 may be attached to and detached from each other using, for example, a snap-fitting cap structure or an adhesive. The lid part 14 allows the inlet 12 to be open in a case where a specimen solution is to be put in, but closes the inlet 12 to eliminate contamination from the outside and to prevent the specimen solution 40 from being evaporated from the inside in cases other than a case where the specimen solution is to be put in. The specimen solution 40 is liquid that is obtained after nucleic acid is extracted from a specimen collected from, for example, a nasal cavity, a pharynx, an oral cavity, an affected area, and the like of a subject.

In the following description, a surface on which the inlet 12 is provided will be referred to as an upper surface of the test container 1 and a side corresponding to the bottom member 1B will be referred to as a lower surface of the test container 1. Here, the upper surface of the body member 1A is the same as the upper surface of the test container 1, the lower surface of the body member 1A is a surface that is in contact with the upper surface of the bottom member 1B, and the lower surface of the bottom member 1B is the same as the lower surface of the test container 1.

The first storage portion 16 is provided such that the inlet 12 is an end surface of an opening, and stores the specimen solution 40 added dropwise from the inlet 12. The shape of the first storage portion 16 is not particularly limited, and a columnar shape, a conical shape, a truncated conical shape, and the like can be arbitrarily selected as the shape of the first storage portion 16.

In this test container 1, the inlet 12 is formed of the opening of the tubular portion 15 that is formed to penetrate the body member 1A in a thickness direction and to protrude from the surface of the body member 1A, and the first storage portion 16 is formed by an inner side portion of the tubular portion 15 in the body member 1A and the bottom member 1B.

The second storage portion 18 is a storage portion that can store liquid, and functions as a reaction section that causes the specimen solution 40 and a reagent 42 to react with each other. The reagent 42 includes an amplification reagent that is used to amplify a nucleic acid sequence as an object to be tested and a probe that is used for determination. The second storage portion 18 is formed by a recessed portion, which is provided on the lower surface of the body member 1A, and the bottom member 1B.

In the present embodiment, the reagent 42 is stored in the second storage portion 18 in advance. However, the reagent 42 has only to be provided between the first storage portion 16 and the second storage portion 18, and does not necessarily need to be stored in the second storage portion 18. The reagent 42 includes an amplification reagent that is used to amplify a specific nucleic acid sequence, a probe that is used to detect a specific nucleic acid sequence, and the like. Examples of the amplification reagent include substrates, such as a primer, polymerase, and dNTP, a salt, and the like. The reagent 42 may further include an additive, such as a reducing agent, a buffer, and the like. In a case where a nucleic acid to be amplified is an RNA, the reagent 42 may further include a reverse transcription primer and a reverse transcriptase. The reagent 42 is appropriately selected according to an amplification method and a detection method. For example, in a case where a specific nucleic acid sequence is detected using a fluorescence method, the reagent 42 may include an amplification reagent and a fluorescent probe. The form of a reagent to be enclosed is not particularly limited, and any liquid or solid reagent can also be used. For example, a powdery reagent prepared from the lyophilization of a liquid reagent, or a reagent shaped into pellets or granules may be enclosed.

The first flow channel 20 connects the first storage portion 16 to the second storage portion 18. The specimen solution 40 put into the first storage portion 16 is fed to the second storage portion 18 through the first flow channel 20. The first flow channel 20 is formed by a linear recessed portion that is formed on the lower surface of the body member 1A to extend from the first storage portion 16 to the second storage portion 18, and the upper surface of the bottom member 1B (see FIGS. 3A, 3B, and 3C). Likewise, each of the second flow channel 24 and the third flow channel 26 is also formed by a linear recessed portion that is formed on the lower surface of the body member 1A, and the upper surface of the bottom member 1B.

The first cylinder 31 of which one end 31b is connected to the first storage portion 16 via the second flow channel 24 is provided such that the other end 31a is open to the outside.

The second cylinder 32 of which one end 32b is connected to the second storage portion 18 via the third flow channel 26 is provided such that the other end 32a is open to the outside.

The first cylinder 31 and the second cylinder 32 are tubular portions formed in a plane direction of the body member 1A, and are formed from an end of the body member 1A toward the inside.

The first plug 33 is provided to be movable in the first cylinder 31. The second plug 34 is provided to be movable in the second cylinder 32. The first plug 33 and the second plug 34 are, for example, rubber plugs, and have functions of blocking outside air in the first cylinder 31 and the second cylinder 32, respectively.

Since a first pressing unit of a pressing machine to be described later can be inserted into the first cylinder 31 from the other end 31a, which is open to the outside, the first plug 33 can be pressed toward an internal space in the first cylinder 31 by the first pressing unit. Likewise, since a second pressing unit of the pressing machine to be described later can be inserted into the second cylinder 32 from the other end 32a, which is open to the outside, the second plug 34 can be pressed toward an internal space in the second cylinder 32 by the second pressing unit. The internal space is under the atmospheric pressure in a state where the plug is not pressed by the pressing machine.

The test container 1 is adapted such that the internal space including the first storage portion 16, the second storage portion 18, the first flow channel 20, the second flow channel 24, and the third flow channel 26 can be pressurized in a case where the first plug 33 and the second plug 34 are pressed and moved from the outside. There is a possibility that gas in the specimen solution 40 and the reagent 42 is generated as foam and the amplification of a nucleic acid is inhibited. It is possible to suppress foaming and to suppress the inhibition of amplification of a nucleic acid, which is caused by foaming, by pressurizing the internal space in a case where the specimen solution 40 and the reagent 42 are mixed with each other in the second storage portion 18 and liquid present in the second storage portion 18 is then heated. For this reason, since a nucleic acid amplification step in the second storage portion 18 can be caused to proceed without being inhibited, it is possible to improve test accuracy without causing a delay in amplification time or insufficient amplification.

In a case where the bottom member 1B of the test container 1 is a film, it is possible to improve a contact property between the second storage portion 18 and a first heating unit 112 and a contact property between the first storage portion 16 and a second heating unit 114 during the heating of the first storage portion 16 and the second storage portion 18 in a test device 100 to be described later. Meanwhile, since the bottom member 1B expands or contract in a case where the first storage portion 16 and the second storage portion 18 are heated to a certain temperature or more, twist occurs. For this reason, a contact property between the bottom member and the heating unit may be reduced and heating efficiency is reduced. On the other hand, in a case where the internal space of the test container 1 is pressurized and appropriately pressurized such that twist does not occur on the bottom member 1B, a contact property between the bottom member 1B and the heating unit can be improved and heating efficiency can be improved.

In a case where the specimen solution 40 is stored in the first storage portion 16 and the inlet 12 is closed by the lid part 14, the internal space of the test container 1 is hermetically sealed by the first plug 33 and the second plug 34. As shown in FIG. 4, the first plug 33 is disposed closer to the other end 31a, which is open to the outside, than the middle of the first cylinder 31 in a length direction in an initial state before liquid is fed. On the other hand, the second plug 34 is disposed closer to one end 32b than the middle of the second cylinder 32 in a length direction. In a case where the first plug 33 is pressed from the outside in the first cylinder 31 (arrow P1) and is moved toward the internal space as shown by a broken line arrow A1 as shown in FIG. 4 in this state, the internal space is pressurized. As a result, the second plug 34 positioned in the second cylinder 32 is interlocked and moved to be pushed out from the internal space to the outside as shown by a broken line arrow A2. Since the second plug 34 is moved while being interlocked with the movement of the first plug 33, the pressure of the internal space is adjusted and the specimen solution 40 stored in the first storage portion 16 can be fed to the second storage portion 18 (arrow B). Since the second plug 34 can be moved while being interlocked with the movement of the first plug 33, liquid can be fed with weak pressing.

“Design Change Example 1”

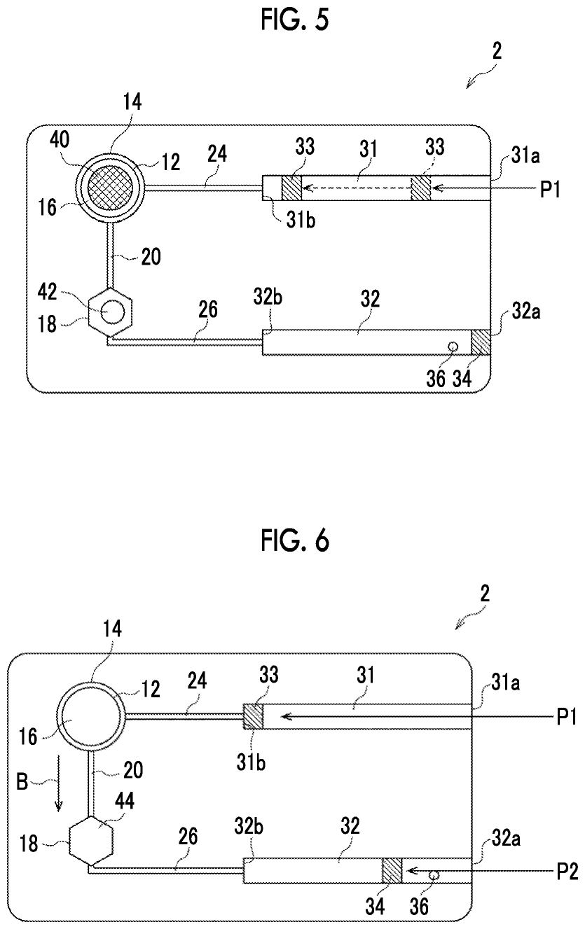

FIG. 5 is a plan view schematically showing a test container 2 of Design Change Example 1 of the test container 1 according to the first embodiment. In Design Change Example and embodiments to be described below, the same elements as the test container 1 according to the first embodiment will be denoted by the same reference numerals as the test container 1 and the detailed description thereof will be omitted. This test container 2 is different from the test container 1 according to the first embodiment in that an air vent 36 is provided at a part of the second cylinder 32.

As shown in FIG. 5, the first plug 33 is disposed closer to the other end 31a, which is open to the outside, than the middle of the first cylinder 31 in a length direction in an initial state before liquid is fed. The second plug 34 is disposed closer to the other end 32a of the second cylinder 32 than the air vent 36. In this case, the internal space is open through the air vent 36. In a case where the first plug 33 is pressed from the outside in the first cylinder 31 (arrow P1) and is moved toward the internal space as shown in FIG. 5 in this state, the specimen solution 40 stored in the first storage portion 16 can be fed to the second storage portion 18 with the movement of the first plug 33. Further, air present in the second cylinder 32 is discharged from the air vent 36 with the movement of the first plug 33.

Further, in a case where the second plug 34 is moved closer to the internal space, that is, the one end 32b of the second cylinder 32 than the air vent 36, a state where the internal space is hermetically sealed can be made. Furthermore, in a case where the first plug 33 and the second plug 34 are pressed from the outside (arrows P1 and P2) as shown in FIG. 6 from the state where the internal space is hermetically sealed, the internal space can be pressurized. Accordingly, even in this test container 2, there is a possibility that gas in the specimen solution 40 and the reagent 42 is generated as foam and the amplification of a nucleic acid is inhibited. It is possible to suppress foaming and to suppress the inhibition of amplification of a nucleic acid, which is caused by foaming, by pressurizing the internal space in a case where the specimen solution 40 and the reagent 42 are caused to react with each other in the second storage portion 18 and liquid present in the second storage portion 18 is heated. For this reason, since a nucleic acid amplification step in the second storage portion 18 can be caused to proceed without being inhibited, it is possible to improve test accuracy without causing a delay in amplification time or insufficient amplification.

“Design Change Example 2”

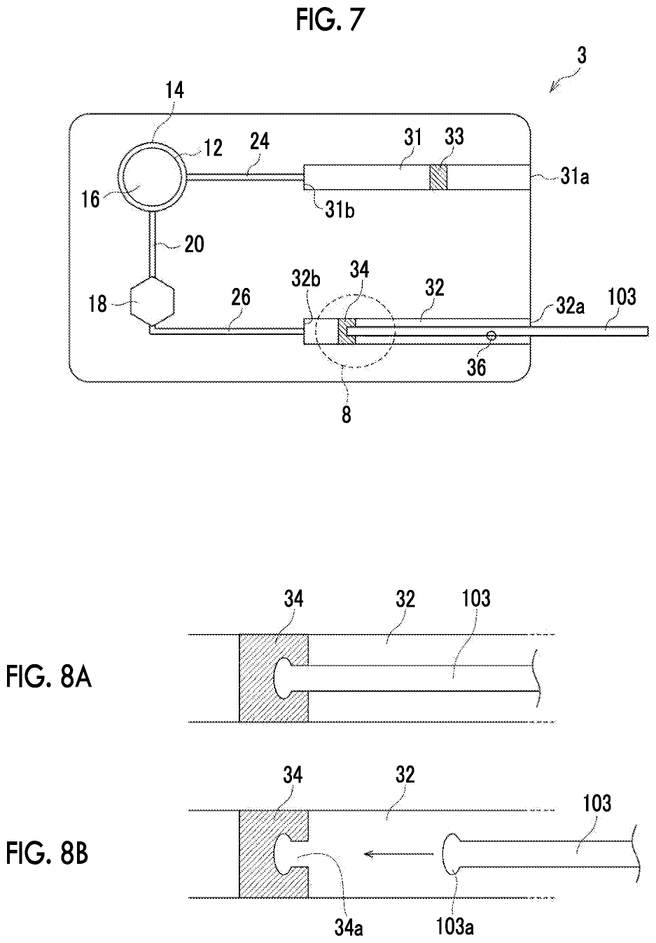

FIG. 7 is a plan view schematically showing a test container 3 of Design Change Example 2 of the test container 1 according to the first embodiment. FIGS. 8A and 8B are enlarged views of a second plug 34 provided in the second cylinder 32, FIG. 8A shows a state where a second push rod 103 of a second pressing unit 104 to be described later is set in the second plug 34 provided in the second cylinder 32 in this test container 3, and FIG. 8B shows a state where the second push rod 103 is not yet set in the second plug 34.

This test container 3 is different from the test container 1 according to the first embodiment in that the second plug 34 includes a recessed hole 34a to which a protrusion 103a formed at a distal end of the second push rod 103 is to be fitted.

In a case where the protrusion 103a of the second push rod 103 and the hole 34a of the second plug 34 are fitted to each other to integrate the second push rod 103 with the second plug 34 as shown in FIGS. 8A and 8B and the second plug 34 is forcibly moved, the pressure of the internal space can be adjusted. The first plug 33 is disposed closer to the other end 31a, which is open to the outside, than the middle of the first cylinder 31 in a length direction in an initial state before liquid is fed. The second plug 34 is disposed closer to the one end 32b than the middle of the second cylinder 32 in a length direction. In a case where the second plug 34 positioned in the second cylinder 32 is moved to the outside (to the right side in FIG. 7) by the second push rod 103 in this state, the specimen solution 40 stored in the first storage portion 16 can be fed to the second storage portion 18 with the movement of the second plug 34. In this case, the first plug 33 positioned in the first cylinder 31 is interlocked and moved to be pulled toward the internal space with the movement of the second plug 34.

The first plug 33 positioned in the first cylinder 31 may also comprise a hole to which a protrusion formed at a distal end of a first push rod 101 is to be fitted and the first push rod 101 and the second push rod 103 may be fitted to the first plug 33 and the second plug 34, respectively, so that the movement of the first cylinder 31 and the movement of the second cylinder 32 may be independently controlled.

Further, in a case where the second plug 34 is pushed toward the internal space by the second push rod 103 and the first plug 33 is also pressed from the outside toward the internal space, the internal space can be pressurized. Accordingly, even in this test container 3, there is a possibility that gas in the specimen solution 40 and the reagent 42 is generated as foam and the amplification of a nucleic acid is inhibited. It is possible to suppress foaming and to suppress the inhibition of amplification of a nucleic acid, which is caused by foaming, by pressurizing the internal space in a case where the specimen solution 40 and the reagent 42 are caused to react with each other in the second storage portion 18 and liquid present in the second storage portion 18 is heated. For this reason, since a nucleic acid amplification step in the second storage portion 18 can be caused to proceed without being inhibited, it is possible to improve test accuracy without causing a delay in amplification time or insufficient amplification.

“Test Container According to Second Embodiment”

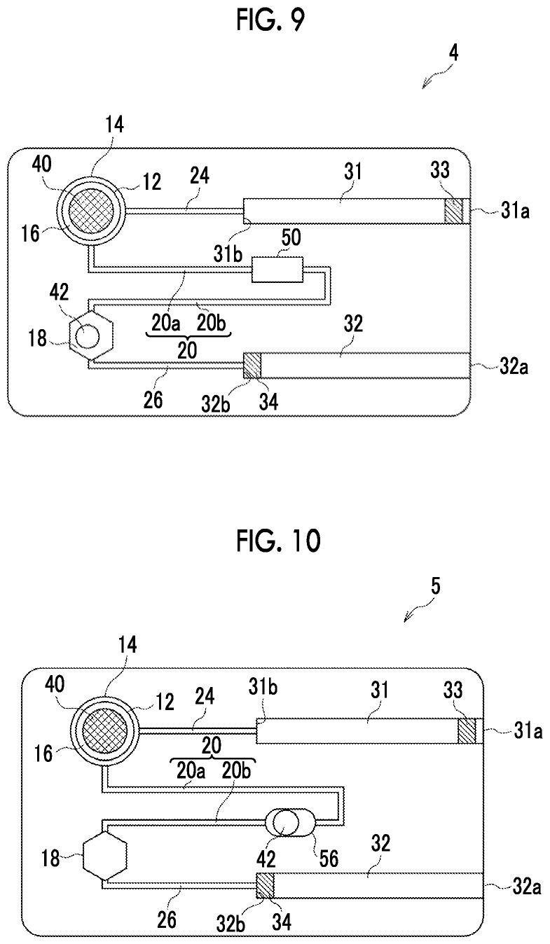

FIG. 9 is a plan view schematically showing a test container 4 according to a second embodiment.

The test container 4 comprises a purification chamber 50 that is provided in the middle of a first flow channel 20 connecting a first storage portion 16 to a second storage portion 18. The first flow channel 20 includes a first flow channel-first portion 20a that connects the first storage portion 16 to the purification chamber 50, and a first flow channel-second portion 20b that connects the purification chamber 50 to the second storage portion 18.

The purification chamber 50 is a chamber that is used to remove impurities from a specimen solution. A purification method is not particularly limited, and a publicly known method can be used. For example, a membrane filter method, an ultrafiltration method, a dialysis method, a gel filtration method, a desalination method, or the like can be used. Further, a method of trapping impurities using an adsorbent, such as an ion exchange resin or a molecular sieve may be used. Since the purification chamber 50 is provided, it is possible to suppress the inhibition of amplification of a nucleic acid, which is caused by impurities included in a specimen solution 40, and to perform a more accurate test.

This test container 4 has the same configuration as the test container 1 according to the first embodiment except for the above-mentioned configuration. Accordingly, the same effect as described above can be obtained.

“Test Container According to Third Embodiment”

FIG. 10 is a plan view schematically showing a test container 5 according to a third embodiment.

The test container 5 comprises a third storage portion 56 that is provided in the middle of a first flow channel 20 connecting a first storage portion 16 to a second storage portion 18. The first flow channel 20 includes a first flow channel-first portion 20a that connects the first storage portion 16 to the third storage portion 56, and a first flow channel-second portion 20b that connects the third storage portion 56 to the second storage portion 18.

A reagent 42 is stored in the third storage portion 56. The reagent 42 comprises, for example, an amplification reagent and a probe for detection. Since the reagent 42 is provided in the middle of the first flow channel 20 connecting the first storage portion 16 to the second storage portion 18, the reagent is dissolved and mixed with the specimen solution while the specimen solution 40 passes through the first flow channel 20. Accordingly, since a nucleic acid can start to be amplified immediately after the specimen solution 40 reaches the second storage portion 18, a time required for an amplification step can be shortened.

This test container 5 has the same configuration as the test container 1 according to the first embodiment except for the above-mentioned configuration. Accordingly, the same effect as described above can be obtained.

“Test Container According to Fourth Embodiment”

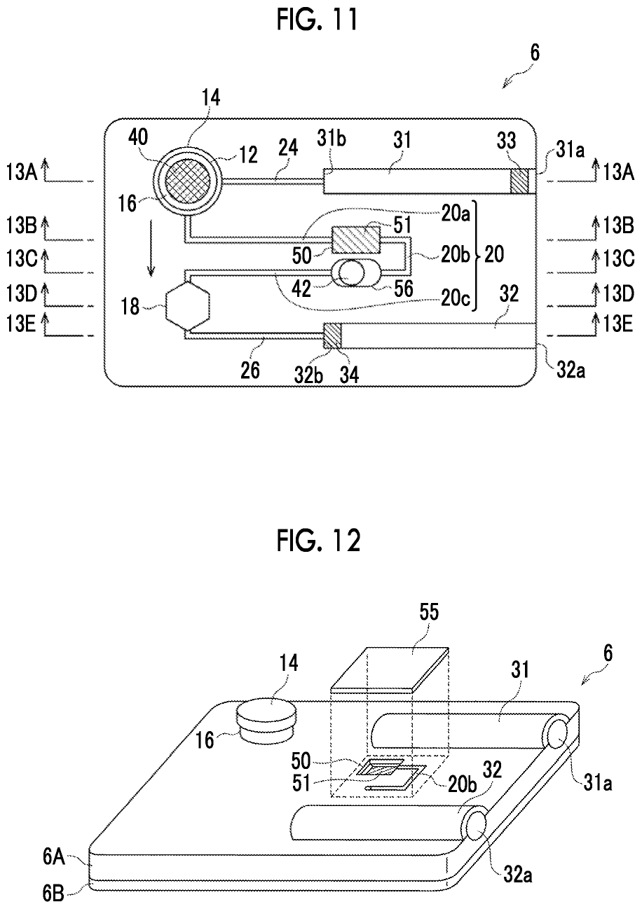

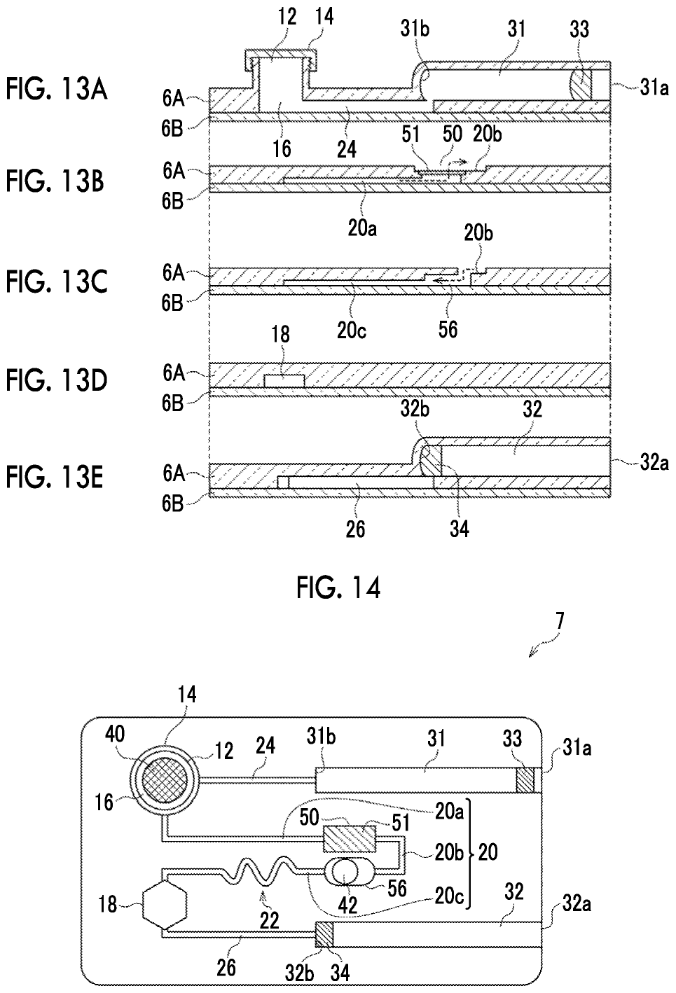

FIG. 11 is a plan view schematically showing a test container 6 according to a fourth embodiment, and FIG. 12 is a perspective view of the test container 6 shown in FIG. 11. Further, among FIGS. 13A, 13B, 13C, 13D, and 13E, FIG. 13A shows a cross section taken along line 13A-13A of FIG. 11, FIG. 13B shows a cross section taken along line 13B-13B of FIG. 11, FIG. 13C shows a cross section taken along line 13C-13C of FIG. 11, FIG. 13D shows a cross section taken along line 13D-13D of FIG. 11, and FIG. 13E shows a cross section take along line 13E-13E of FIG. 11.

The test container 6 according to the present embodiment includes a body member 6A in which a recessed portion and a hole portion forming a part of a flow channel structure including flow channels and storage portions are formed, and a bottom member 6B that forms a bottom surface of the flow channel. The body member 6A and the bottom member 6B of the present embodiment are made of the same materials as the body member 1A and the bottom member 1B of the test container 1.

Like the test container 1, this test container 6 comprises an inlet 12, a lid part 14, a first storage portion 16, a second storage portion 18, a first flow channel 20, a first cylinder 31, a second cylinder 32, a first plug 33, and a second plug 34. The test container 6 further comprises a second flow channel 24 and a third flow channel 26. The test container 6 further comprises a purification chamber 50 and a third storage portion 56 that are provided in the middle of the first flow channel 20 and are arranged in order from the first storage portion 16. The first flow channel 20 includes a first flow channel-first portion 20a that connects the first storage portion 16 to the purification chamber 50, a first flow channel-second portion 20b that connects the purification chamber 50 to the third storage portion 56, and a first flow channel-third portion 20c that connects the third storage portion 56 to the second storage portion 18.

The purification chamber 50 is formed by a hole that is provided near the center of the body member 6A and penetrates the body member 6A in a thickness direction, and the bottom member 6B; and a purification filter 51 is provided in the middle of the hole, which forms the purification chamber 50, in the thickness direction of the body member 6A.

The third storage portion 56 is a storage portion that stores a reagent 42, and is formed by a recessed portion that is provided at a position adjacent to the purification chamber 50 on the lower surface of the body member 6A, and the bottom member 6B (see FIGS. 12 and 13C).

The first flow channel-first portion 20a is formed by a recessed portion that is formed on the lower surface of the body member 6A to extend from the first storage portion 16 to the purification chamber 50, and the bottom member 6B. The first flow channel-first portion 20a communicates with the first storage portion 16 and the purification chamber 50 on the lower surface of the body member 6A.

The first flow channel-second portion 20b is formed by a recessed portion that is formed on the upper surface of the body member 6A to extend from the purification chamber 50 to the third storage portion, and a sealing member 55 that covers the purification chamber and the first flow channel-second portion 20b open to the upper surface. After the purification filter 51 is inserted into the purification chamber 50, the sealing member 55 covers the purification chamber 50 and the first flow channel-second portion 20b and is fixed to the upper surface of the body member 6A. The first flow channel-second portion 20b communicates with the purification chamber 50 and the third storage portion 56 on the upper surface of the body member 6A.

The first flow channel-third portion 20c is formed by a recessed portion that is formed on the lower surface of the body member 6A to extend from the third storage portion 56 to the second storage portion 18, and the bottom member 6B. The first flow channel-third portion 20c communicates with the third storage portion 56 and the second storage portion 18 on the lower surface of the body member 6A.

According to the above-mentioned configuration, the specimen solution 40 stored in the first storage portion 16 is fed to the second storage portion 18 along the following route. First, the specimen solution 40 passes through the first flow channel-first portion 20a, which is provided on the lower surface of the body member 6A, from the first storage portion 16 and flows into the purification chamber 50, which communicates with the first flow channel-first portion 20a, from the lower surface of the body member 6A. As shown in FIG. 13B by a broken line arrow, the specimen solution 40 flowing into the purification chamber 50 passes through the purification filter 51 in the purification chamber 50 and flows into the first flow channel-second portion 20b that communicates with the purification chamber 50 on the upper surface of the body member 6A. As shown in FIG. 13C by a broken line arrow, the specimen solution 40 flowing into the first flow channel-second portion 20b flows into the third storage portion 56 from an upper portion of the third storage portion 56 with which the first flow channel-second portion 20b communicates. Further, the specimen solution 40 passes through the first flow channel-third portion 20c that communicates with the third storage portion 56 on the lower surface of the body member 6A, and is fed to the second storage portion 18.

The first cylinder 31, the second cylinder 32, the first plug 33, and the second plug 34 of this test container 6 have the same configuration and the same functions as those of the test container 1 according to the first embodiment. Accordingly, the same effect as the test container 1 can be obtained.

Further, since the purification chamber 50 is provided, it is possible to suppress the inhibition of amplification of a nucleic acid, which is caused by impurities included in the specimen solution 40, and to perform a more accurate test.

Furthermore, since the reagent 42 is provided downstream of the purification chamber 50 in the middle of the first flow channel 20 that connects the first storage portion 16 to the second storage portion 18, the reagent 42 is dissolved and mixed with the specimen solution while a specimen solution 40 passes through the first flow channel 20. Accordingly, since a nucleic acid can start to be amplified immediately after the specimen solution 40 reaches the second storage portion 18, a time required for an amplification step can be shortened.

“Test Container According to Fifth Embodiment”

FIG. 14 is a plan view schematically showing a test container 7 according to a fifth embodiment.

The test container 7 has configuration in which the first flow channel-third portion 20c between the third storage portion 56 and the second storage portion 18 comprises a stirring flow channel 22 in the test container 6 according to the fourth embodiment. In the present embodiment, the stirring flow channel 22 is a bellows-shaped flow channel. However, the stirring flow channel 22 may be adapted to be capable of generating turbulence, and may have configuration in which, for example, a baffle plate is provided in a linear flow channel.

Since the stirring flow channel 22 is provided between the third storage portion 56 and the second storage portion 18, it is possible to cause the dissolution of the reagent 42 to proceed and to facilitate the mixing of the specimen solution 40 and the reagent 42. Further, this test container 7 has the same configuration as the test container 6 according to the fourth embodiment except for the above-mentioned configuration. Accordingly, the same effect as the test container 6 can be obtained.

“Test Device”

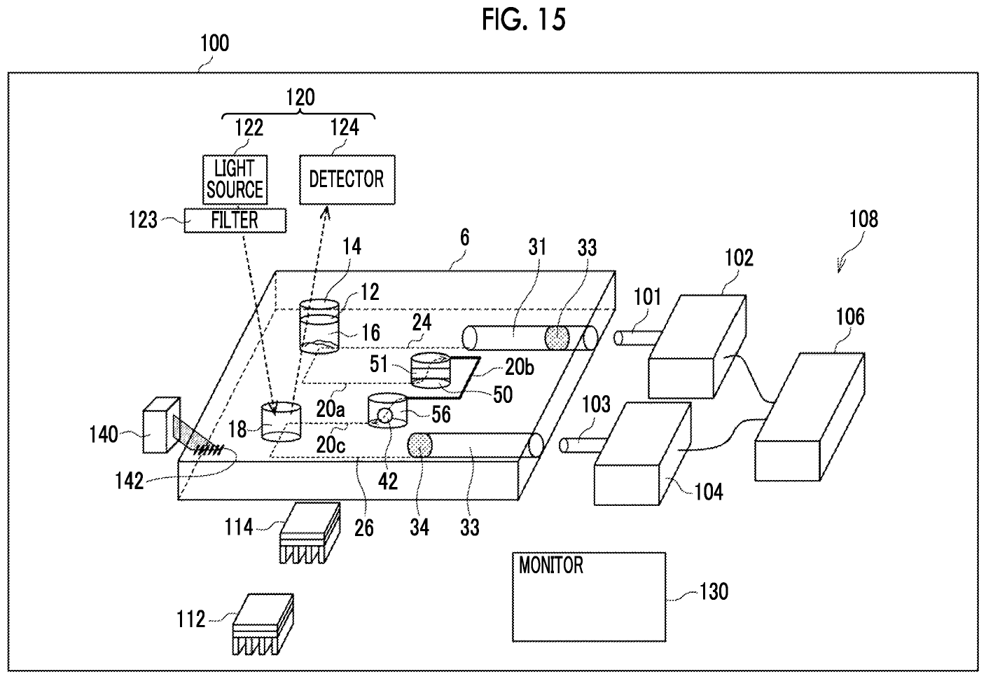

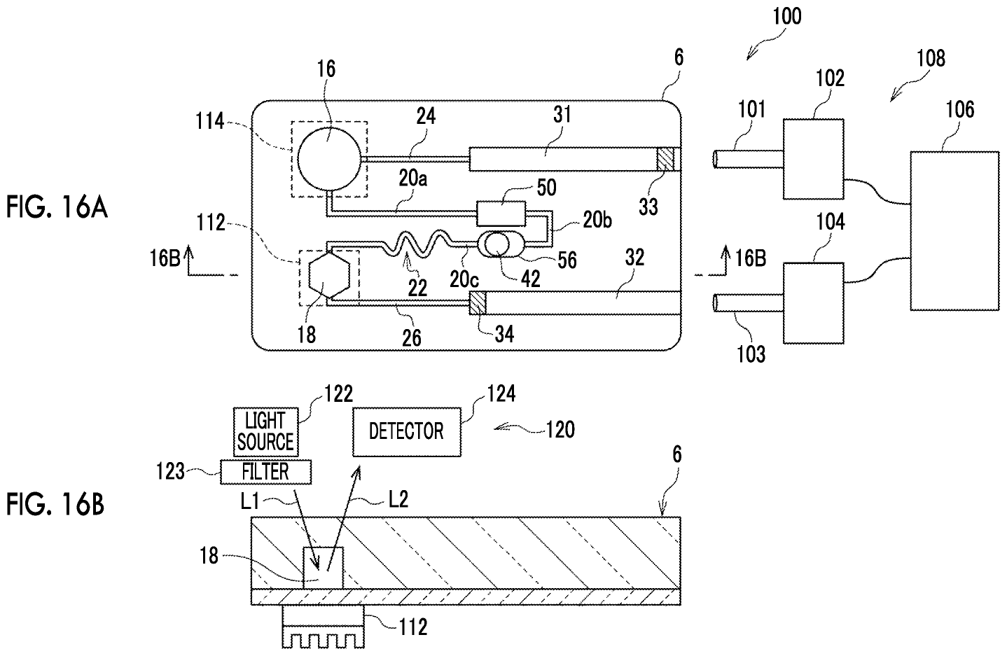

FIG. 15 is a diagram showing the schematic configuration of a test device 100 according to an embodiment. This test device 100 comprises a test container 6, the pressing machine 108, a first heating unit 112, a second heating unit 114, a detection unit 120, a monitor 130, and an identification (ID) management unit 140. FIG. 16A is a plan view showing a positional relationship between the test container 6 and the pressing machine 108 of the test device 100. Further, FIG. 16B is a cross-sectional view that is taken along line 16B-16B of FIG. 16A and shows a positional relationship between the test container 6 and the detection unit 120 of the test device 100. A horizontal plane of the test container 6 may coincide with or be inclined with respect to a horizontal plane of the test device 100, or may be oriented in a vertical direction.

The test device 100 comprises the test container 6 according to the fourth embodiment in this configuration, but any of the test containers 1 to 7 may be used.

The pressing machine 108 comprises a first pressing unit 102 that comprises a first push rod 101, a second pressing unit 104 that comprises a second push rod 103, and a pressing control unit 106 that controls the first pressing unit 102 and the second pressing unit 104. The first pressing unit 102 and the second pressing unit 104 can push in or pull out the first push rod 101 and the second push rod 103 with actuators that uses stepping motors, solenoids, or the like. The actuator may be adapted to use power such as pneumatic pressure.

The first pressing unit 102 is disposed at a position where the first push rod 101 can be inserted into the first cylinder 31 from the other end 31a open to the outside of the first cylinder 31 in a state where the test container 6 is installed. The first plug 33 can be pressed and moved in the first cylinder 31 toward the internal space by the first push rod 101.

The second pressing unit 104 is disposed at a position where the second push rod 103 can be inserted into the second cylinder 32 from the other end 32a open to the outside of the second cylinder 32 in a state where the test container 6 is installed. The second plug 34 can be pressed and moved in the second cylinder 32 toward the internal space by the second push rod 103.

In a case where the first plug 33 is pressed and moved toward the internal space by the first pressing unit 102 in a state where the internal space is hermetically sealed after a specimen solution 40 is put into the first storage portion 16 of the test container 6 and the lid part 14 is closed, the specimen solution 40 stored in the first storage portion 16 of the test container 6 can be fed to the second storage portion 18.

Further, in a case where the first plug 33 is pressed by the first pressing unit 102 and the second plug 34 is pressed by the second pressing unit 104, the internal space of the test container 6 can be pressurized.

The first heating unit 112 is provided at a position that allows the first heating unit 112 to be in contact with the bottom surface of the second storage portion 18 of the test container 6. The first heating unit 112 heats liquid stored in the second storage portion 18. Here, the liquid stored in the second storage portion 18 is a mixed liquid of the specimen solution 40 and the reagent 42. The first heating unit 112 heats the mixed liquid of the specimen solution 40 and the reagent 42 to facilitate the amplification of a nucleic acid.

The second heating unit 114 is provided at a position that allows the second heating unit 114 to be in contact with the bottom surface of the first storage portion 16 of the test container 6. The second heating unit 114 heats liquid stored in the first storage portion 16. Here, the liquid stored in the second storage portion 18 is the specimen solution 40. The second heating unit 114 heats the specimen solution 40 for pretreatment. The test device 100 may not comprise the second heating unit 114 in a case where the heating of the specimen solution 40 for pretreatment is not required.

The first heating unit 112 comprises a Peltier element or the like and is adapted to be capable of controlling a temperature, and performs a temperature cycle in the amplification step. On the other hand, the second heating unit 114 does not require the temperature cycle performed by the first heating unit 112, and is formed of, for example, a heater. A publicly known heating mechanism can be used as a heating mechanism used for each of the first heating unit 112 and the second heating unit 114, and the heating mechanism is not particularly limited.

The detection unit 120 detects whether or not an object to be detected is included in the specimen solution 40 in the second storage portion 18. The detection unit 120 comprises an excitation light source 122, a wavelength selective filter 123, and a photodetector 124. The detection unit 120 is disposed above the second storage portion 18 of the test container 6. The excitation light source 122 irradiates the inside of the second storage portion 18 with excitation light L1 having a specific wavelength through the wavelength selective filter 123. The photodetector 124 detects fluorescence L2 that is excited by the excitation light L1 and is generated from a fluorescent probe. The excitation light L1 is selected according to an excitation wavelength of the fluorescent probe. Further, the detection unit 120 may include a filter that adjusts intensity or the amount of light, a lens that is used to converge the excitation light L1 or to condense the fluorescence L2 generated from a detection probe to the photodetector 124, an optical system, or the like, as necessary.

An LED, a laser, or the like is used as the excitation light source 122. The wavelength selective filter 123 is a filter that transmits only light having a wavelength corresponding to the excitation wavelength of the probe of the light emitted from the excitation light source 122. For example, a photodiode, a photomultiplier, or the like is applied as the photodetector 124.

The monitor 130 is, for example, a touch panel display, and starts measurement or displays test results in a case where a touch panel is operated.

The ID management unit 140 comprises a bar code reader that reads out a bar code 142 provided on the test container 6, and manages the ID of the test container 6.

“Nucleic Acid Test Method”

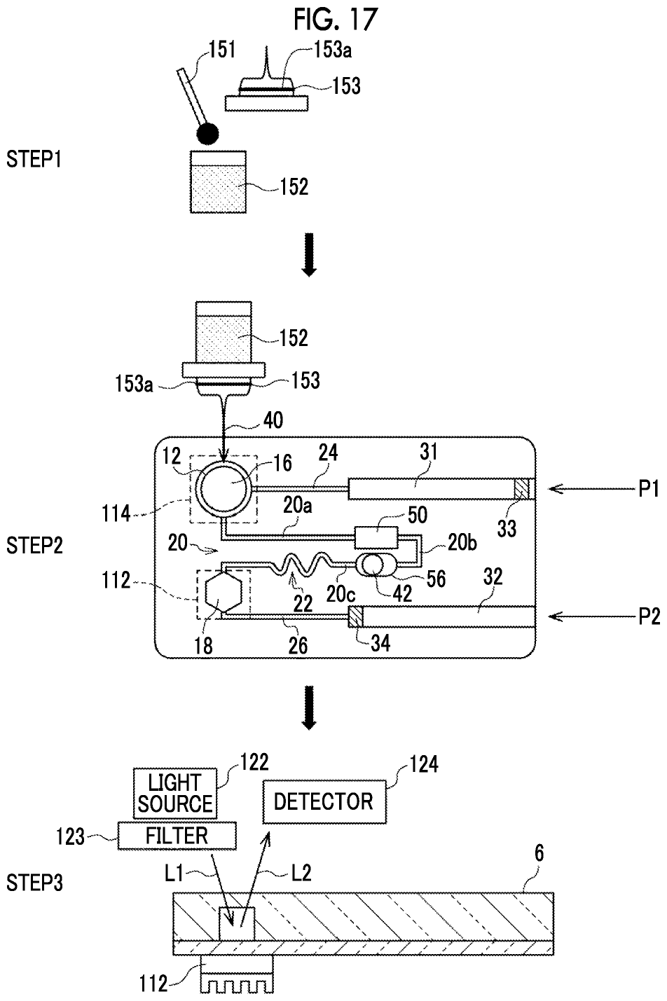

A nucleic acid test method according to an embodiment using the test device 100 according to the embodiment will be described with reference to FIG. 17.

This nucleic acid test method includes a nucleic acid extraction step (STEP1), an amplification step (STEP2), and a detection step (STEP3). The nucleic acid extraction step of STEP1 is performed outside the test device 100, and the amplification step and the detection step are performed in the test device 100.

(Nucleic Acid Extraction Step)

First, a specimen is collected from a living body using a collection tool 151, such as a swab, prepared separately from the test device 100. Specifically, a specimen is collected from a nasal cavity, a pharynx, the inside of an oral cavity, an affected area, or the like of a subject using the collection tool. Alternatively, body fluid, such as lavage fluid in a nasal cavity, a pharynx, or an oral cavity, saliva, urine, or blood, is collected as a specimen.

Then, a nucleic acid, such as DNA or RNA, is extracted from a specimen using an extraction tool 152 prepared separately from the test device 100 and is brought into the state of a specimen solution 40. In the present embodiment, the extraction tool 152 stores a nucleic acid extraction solution and the specimen is immersed in the nucleic acid extraction solution to extract a nucleic acid. A publicly known nucleic acid extraction method can be used as a nucleic acid extraction method without particular limitation. Examples of the nucleic acid extraction method include a method using a surfactant or a chaotropic substance and a method of applying physical shear, such as an ultrasonic wave or a bead mill.

(Amplification Step)

A dropping cap 153 comprising a coarse filter 153a, which removes coarse materials, is mounted on the extraction tool 152, and the specimen solution 40 is put in from the inlet 12 of the test container 6. The specimen solution 40 may be sucked from the extraction tool 152 with a pipette or the like and may be put in from the inlet 12. After the specimen solution 40 is completely put in, the inlet 12 is closed by the lid part 14 and the internal space of the test container 6 is hermetically sealed.

The test container 6 is installed in a test container installation portion of the test device 100, and the test device 100 performs the following amplification step and the following detection step.

The specimen solution 40 stored in the first storage portion 16 is heated using the second heating unit 114. Heating can facilitate the elution of a nucleic acid or suppress the decomposition of a nucleic acid extracted via the inactivation of restriction enzyme. A heating temperature may be a temperature range that does not adversely affect a nucleic acid, and it is preferable that the heating temperature is in a range of, for example, about 50° C. to 95° C.

The specimen solution 40 subjected to heating treatment as pretreatment in the first storage portion 16 is fed to the second storage portion 18. The first push rod 101 of the first pressing unit 102 is inserted from the other end 31a open to the outside the first cylinder 31 to press and move the first plug 33 toward the internal space of the test container 6. Accordingly, the specimen solution 40 stored in the first storage portion 16 can be fed to the second storage portion 18. In this case, the first plug 33 is pushed in and the internal space is pressurized, so that the second plug 34 positioned in the second cylinder 32 is moved to the outside. Therefore, pressure in the internal space is adjusted and liquid can be fed with weak pressing.

The specimen solution 40 is fed to the second storage portion 18 from the first storage portion 16 via the purification chamber 50 and the third storage portion 56. Impurities contained in the specimen solution 40 are removed by the purification filter 51 in the purification chamber 50, and the specimen solution 40 from which impurities have been removed is fed to the third storage portion 56. Since a reagent 42 is provided in the third storage portion 56, the reagent 42 is dissolved in a case where the specimen solution 40 flows into the third storage portion 56. Accordingly, the specimen solution 40 and the reagent 42 are fed to the second storage portion 18 while being mixed with each other.

After a mixed liquid of the specimen solution 40 and the reagent 42 is fed to the second storage portion 18, the second push rod 103 of the second pressing unit 104 is inserted from the other end 32a open to the outside of the second cylinder 32, presses the second plug 34, and pressurizes the internal space. In this pressurized state, the mixed liquid present in the second storage portion 18 is heated by the first heating unit 112 and a specific nucleic acid sequence is amplified. An amplification method is not limited, but, for example, an RT-PCR method or a PCR method is used. In a case where a PCR method is used, a step of dissociating a double-stranded DNA into a single-stranded DNA at a high temperature (thermal denaturation step), a step of lowering a temperature and binding a primer to the single-stranded DNA (annealing step), and a step of newly synthesizing a double-stranded DNA by polymerase using the single-stranded DNA as a template (elongation step) are repeated. Examples of a temperature cycle of the thermal denaturation step, the annealing step, and the elongation step include 20 to 50 repetitions of one cycle of the thermal denaturation step performed for 1 minute at a temperature of 94° C., the annealing step performed for 1 minute at a temperature of 50 to 60° C., and the elongation step performed for 1 to 5 minutes at a temperature of 72° C. Further, the annealing step and the elongation step may be performed at one temperature.

Examples of such a temperature cycle include 20 to 50 repetitions of one cycle of the thermal denaturation step and the annealing step performed for 1 minute at a temperature of 94° C. and the elongation step performed for 1 minute at a temperature of 60° C. The temperature and time of the temperature cycle of the amplification step are not particularly limited and are arbitrarily selected depending on the performance of polymerase or a primer.

(Detection Step)

Fluorescence detection is performed for each cycle of the above-mentioned temperature cycle, and an amplification situation is monitored in real time. That is, the amplification step and the detection step are performed in parallel in the present embodiment. Results of the fluorescence detection are displayed on the monitor 130.

In a case where a specific nucleic acid sequence is present in the specimen solution 40, the nucleic acid sequence is amplified in the amplification step and a fluorescent probe labeled with this specific nucleic acid sequence is irradiated with excitation light, so that fluorescence is detected. On the other hand, in a case where a specific nucleic acid sequence is not present in the specimen solution 40, fluorescence is not detected even though excitation light is applied. Accordingly, it is possible to determine whether or not a specific nucleic acid sequence is present.

According to the test method using this test device 100, it is possible to pressurize the internal space by pressing the first plug 33 and the second plug 34 from the outside (arrows P1 and P2) in a state where the internal space of the test container 6 is hermetically sealed. Then, since the mixed liquid of the specimen solution 40 and the reagent 42 is heated using the first heating unit 112 in the pressurized state, it is possible to suppress foaming, which may be caused during heating, and to suppress the inhibition of amplification of a nucleic acid that is caused by foaming. For this reason, since a nucleic acid amplification step in the second storage portion 18 can be caused to proceed without being inhibited, it is possible to improve test accuracy without causing a delay in amplification time or insufficient amplification.

In the test method using the test device 100, whether or not a nucleic acid is present is determined by a fluorescence method using a fluorescent probe. However, a method of determining whether or not a nucleic acid is present is not limited to the fluorescence method, and other detection methods, such as a nucleic acid chromatography method, a light scattering method, a sequencing method, and an electrochemical method, may be used. It is possible to realize these methods by appropriately changing the detection unit. In addition, a detection method using a fluorescence method or an electrochemical method is particularly preferable from the viewpoint that detection can be performed in real time and a strong positive patient can be quickly determined.

What is claimed is:

1. A test container comprising:
an inlet into which a specimen solution is to be put;
an attachable and detachable lid part that covers the inlet;
a first storage portion that is provided such that the inlet is an end surface of an opening and that stores a specimen solution added dropwise from the inlet;
a second storage portion that is capable of storing liquid and causes the specimen solution and a reagent to react with each other;
a first flow channel that connects the first storage portion to the second storage portion;
a first cylinder of which one end is connected to the first storage portion via a second flow channel and the other end is open to an outside;
a second cylinder of which one end is connected to the second storage portion via a third flow channel and the other end is open to an outside;
a first plug that is provided to be movable in the first cylinder; and
a second plug that is provided to be movable in the second cylinder,
wherein an internal space including the first storage portion, the second storage portion, the first flow channel, the second flow channel, and the third flow channel is capable of being pressurized in a case where the first plug and the second plug are pressed and moved from the outside.

2. The test container according to claim 1,
wherein the internal space is hermetically sealed by the first plug and the second plug, and the second plug is moved from the internal space to the outside in the second cylinder while being interlocked with movement of the first plug in a case where the first plug is pressed from the outside and moved toward the internal space in the first cylinder.

3. The test container according to claim 1,
wherein an air vent is provided at a part of the second cylinder and the internal space is capable of being pressurized in a case where the second plug is moved closer to the internal space than the air vent.

4. The test container according to claim 1, further comprising:
a purification chamber that is provided in a middle of the first flow channel and removes impurities in the specimen solution.

5. The test container according to claim 1,
wherein the reagent is stored in the second storage portion.

6. The test container according to claim 1, further comprising:
a third storage portion that is provided in a middle of the first flow channel and stores the reagent.

7. The test container according to claim 6, further comprising:
a purification chamber that is provided in a middle of the first flow channel and removes impurities in the specimen solution,
wherein the third storage portion is provided between the purification chamber and the second storage portion in the middle of the first flow channel.

8. The test container according to claim 6, further comprising:
a stirring flow channel that is provided between the third storage portion and the second storage portion and facilitates mixture of the specimen solution and the reagent.

9. The test container according to claim 1,
wherein a bottom surface of the second storage portion is formed of a film.

10. The test container according to claim 1,
wherein the reagent includes an amplification reagent that amplifies a specific nucleic acid sequence and a probe for determination of a nucleic acid sequence.

11. A test device comprising:
the test container according to claim 1; and
a pressing machine including a first pressing unit that presses the first plug positioned in the first cylinder of the test container from the outside and a second pressing unit that presses the second plug positioned in the second cylinder from the outside,
wherein in a case where the first plug is pressed and moved toward the internal space by the first pressing unit, the specimen solution stored in the first storage portion of the test container is fed to the second storage portion.

12. The test device according to claim 11, further comprising:
a first heating unit that is provided at a position allowing the first heating unit to be in contact with a bottom surface of the second storage portion of the test container and heats liquid stored in the second storage portion.

13. The test device according to claim 11, further comprising:
a second heating unit that is provided at a position allowing the second heating unit to be in contact with a bottom surface of the first storage portion of the test container and heats liquid stored in the first storage portion.

14. The test device according to claim 11, further comprising:
a detection unit that detects whether or not an object to be detected is included in the specimen solution in the second storage portion.

15. The test device according to claim 14,
wherein the reagent in the test container includes an amplification reagent that amplifies a specific nucleic acid sequence and a fluorescent probe for determination of a nucleic acid sequence, and
the detection unit includes an excitation light source that irradiates liquid stored in the second storage portion with excitation light for exciting the fluorescent probe, and a photodetector that detects fluorescence emitted from the fluorescent probe excited by irradiation with the excitation light.

16. A nucleic acid test method using the test device according to claim 15, the method comprising:
immersing a specimen, which is collected from a living body using a collection tool, in a nucleic acid extraction solution to extract a nucleic acid from the specimen;
putting liquid, which includes the nucleic acid, as the specimen solution from the inlet of the test container;
hermetically sealing the inlet by the lid part;
feeding the specimen solution, which is stored in the first storage portion, to the second storage portion by the pressing machine;
amplifying the specific nucleic acid sequence by controlling a temperature of mixed liquid of the specimen solution and the reagent in the second storage portion;
irradiating the mixed liquid with the excitation light and detecting fluorescence generated from the fluorescent probe using the photodetector; and
determining whether or not the specific nucleic acid sequence is present.
